(19) United States
(12) Patent Application Publication
(10) Pub. No.: US 2009/0111736 A1
(43) Pub. Date: Apr. 30, 2009

(54) Orally-Absorbed Solid Dose Formulation for Vancomycin

(75) Inventor: Gita Natarajan Shankar, Saratoga, CA (US)

Correspondence Address: RICHARD ARON OSMAN, 4070 CALLE ISABELLA, SAN CLEMENTE, CA, 92672, US

(73) Assignee: SRI International

(21) Appl. No.: 11/927,579

(22) Filed: Oct. 29, 2007

(51) Int. Cl. A61K 38/14; A61P 31/04

(52) U.S. Cl. 514/8

(57) ABSTRACT

An orally bioavailable pharmaceutical composition comprises at least 40% (w/w) vancomycin; a permeation enhancer component comprising 0.1 to 10.0% (w/w) of a polyoxyethylene sorbitan fatty acid ester; and a particulate carrier onto which the permeation enhancer component is adsorbed.

### STATEMENT REGARDING FEDERALLY SPONSORED RESEARCH

This invention was made with government support under contract numbers N01-AI-05414 awarded by the National Institute of Allergy and Infectious Diseases. The government has certain rights in this invention.

### BACKGROUND OF THE INVENTION

The field of the invention is orally bioavailable formulations for vancomycin.

Vancomycin (VCM) is a tricyclic glycopeptide antibiotic with molecular formula C₆₆H₇₄ClN₉O₂₄. It has a relatively high molecular weight (about 1500 Daltons). When administered by injection or infusion, VCM is indicated for the treatment of serious or severe infections caused by susceptible strains of methicillin-resistant (beta-lactam resistant) staphylococci, for penicillin-allergic patients and patients who cannot receive or who have failed to respond to other drugs, including the penicillins or cephalosporins, and for infections caused by VCM-susceptible organisms that are resistant to other antimicrobial drugs. VCM is also given by mouth to treat intestinal infections, in particular, pseudomembranous colitis caused by Clostridium difficile and staphylococcal enterocolitis.

Enhanced systemic absorption and/or bioavailability of poorly permeable drugs may be achieved by increasing their trans-epithelial and/or paracellular permeation across the gastrointestinal tract by using permeation enhancers or other strategies (see e.g. Aungst, J Pharm Sci (2000) 89:429-42; Cornaire et al., Int J Pharm (2004) 278:119-31; Aungst et al., J Control Release (1996) 41:19-31). VCM is soluble in water and has poor oral absorption with absolute bioavailability of less than 5% in rats without any added absorption enhancer or enzyme inhibitor (Geary and Schlameus (1993) J Control Release 23:65-74). When VCM formulations containing permeation enhancers or surfactants were administered in situ, directly to segments of rat intestine and colon, increased absorption of VCM was reported in lower intestinal segments and colon (Geary and Schlameus et al., supra; Kajita et al., J Pharm Sci (2000) 89:1243-52; and Prasad et al., Int. J. Pharm (2003) 250:181-90). About 30% absolute bioavailability was also reported in an in vivo study in rats after oral administration of a water-in-oil-in-water (w/o/w) emulsion, where VCM was incorporated within an inner aqueous phase of the multiple emulsions. (Shively and Thompson Int. J. Pharm (1995) 117:119-22).

### SUMMARY OF THE INVENTION

One aspect of the invention is an orally bioavailable pharmaceutical composition comprising: a) at least 40% (w/w) vancomycin; b) a permeation enhancer component comprising 0.1 to 10.0% (w/w) of a polyoxyethylene sorbitan fatty acid ester; and c) a particulate carrier onto which the permeation enhancer component is adsorbed, wherein the permeation enhancer component increases the vancomycin apparent permeability coefficient across rat jejunal tissue in mucosal-to-serosal direction as measured in an in vitro Ussing system by at least 25%.

In various embodiments, the carrier is selected from the group consisting of starch, magnesium carbonate, kaolin, colloidal silica, silicon-dioxide, crosslinked polyvinylpyrrolidone and calcium carbonate.

In one embodiment the permeation enhancer component further comprises a P-glycoprotein inhibitor selected from 1 to 20% (w/w) d-alpha-tocopheryl polyethylene glycol 1000 succinate (TPGS) and 0.1 to 10% (w/w) quinidine.

In another embodiment, the permeation enhancer component further comprises 1-20% (w/w) macrogolglycerides, such as lauroyl macrogolglycerides, stearoyl macrogolglycerides, or caprylocaproyl macrogolglycerides.

In one embodiment, the permeation enhancer component further comprises 0.1 to 15% (w/w) of a medium chain fatty acid selected from the group consisting of sodium decanoate, sodium laurate, sodium caprylate.

In another embodiment, the permeation enhancer component further comprises 0.5 to 2.0% (w/w) of a bile salt selected from the group consisting of sodium glycocholate, sodium deoxycholate, sodium taurocholate, sodium fusidate, sodium glycodeoxycholate, and sodium taurodihydrofusidate.

The composition of the invention is preferably in an enteric-coated unit dosage form, wherein the enteric coating preferably dissolves at approximately pH of 6.0 and above.

Another aspect of the invention is a pharmaceutical composition in an oral dosage form, said composition comprising: at least 40% (w/w) vancomycin; and 5-30% (w/w) bile salts, wherein the composition has a vancomycin bioavailability of at least 40% when orally administered to a mammal.

Another aspect of the invention is methods for treating a pathologic microbial infection in a mammal, the method comprising orally administering to the mammal an effective amount of a composition of the invention.

In one embodiment, prior to the administering step, the mammal is administered a P-glycoprotein inhibitor in an amount effective to inhibit P-glycoprotein-mediated efflux of the vancomycin.

Another aspect of the invention are kits comprising a composition of the invention, optionally with a P-glycoprotein inhibitor in a dosage form separate from the composition.

### DETAILED DESCRIPTION OF THE INVENTION

The invention provides compositions, kits, and methods for oral treatment of staphylococci infection and other conditions amenable to systemic treatment with vancomycin. One aspect of the invention is a composition comprising vancomycin, a permeation enhancer component comprising a polyoxyethylene sorbitan fatty acid ester, and a particulate carrier onto which the permeation enhancer component is adsorbed, wherein the permeation enhancer component increases the vancomycin apparent permeability coefficient across jejunal tissue. References herein to vancomycin are intended to include vancomycin and its pharmaceutically-acceptable salts (e.g. vancomycin hydrochloride, etc.). The composition preferably comprises at least 25% vancomycin, and preferably at least 30%, 35%, 40%, 45%, 50%, 55%, 60%, 65%, 70%, or 75% vancomycin. The permeation enhancer component preferably comprises less than 50% of the composition, and more preferably less than 45%, 40%, 35%, 30%, 25%, or 20% of the composition. Most preferably, the vancomycin comprises at least 40% of the composition, and the permeation enhancer component comprises less than 20% of the composition. As used herein, a percentage (%) of the composition refers to weight % (also abbreviated “% (w/w)”) unless indicated otherwise. The permeation enhancer component increases the vancomycin apparent permeability coefficient (Pₐₚₚ) across jejunal tissue by at least 25%, and preferably at least 30%, 35%, 40%, 45%, or 50%. The permeability coefficient is determined across rat jejunal tissue in mucosal-to-serosal direction as measured in an in vitro Ussing system using methods and calculations described in Example 1.

The permeation enhancer component of the composition comprises polyoxyethylene sorbitan fatty acid esters, preferably in amounts of 0.1 to 10.0%, and more preferably 0.1 to 5%. Suitable polyoxyethylene sorbitan fatty acid esters include polyoxyethylene 20 sorbitan monolaurate (polysorbate 20), polyoxyethylene 20 sorbitan monopalmitate (polysorbate 40), polyoxyethylene 20 sorbitan monostearate (polysorbate 60), and polyoxyethylene 20 sorbitan monooleate (polysorbate 80).

The permeation enhancer component may comprise one or more additional ingredient that further increase vancomycin bioavailability. Suitable enhancers are selected from medium-chain glycerides (e.g. glyceryl monooleate, glyceryl monolinoleate, etc.), macrogolglycerides, polyglycols, glycerol esters of fatty acids, pegylated alcoholic esters of fatty acids, glyceryl monoesters, propylene glycol monoesters, medium chain fatty acids, chitosan and chitosan derivatives (see e.g. Cano-Cebrian et al., Curr Drug Deliv. (2005) 2:9-22), and mixtures thereof.

In a preferred embodiment, the permeation enhancer component further comprises 1-20%, and preferably 2-10% macrogolglycerides. Particularly preferred macrogolglycerides are lauroyl macrogol-32 glycerides and steroyl macrogol glycerides, sold as GELUCIRE® 44/14 and GELUCIRE® 50/13 (Gattefosse Corporation, Paramus, N.J.), respectively. Other macrogolglycerides that may be used are caprylocaproyl macrogol-8 glycerides, sold as LABRASOL® (Gattefosse Corporation, Paramus, N.J.). In a preferred embodiment, the permeation enhancer component comprises 1-20%, 2-10%, or 4-6% lauroyl macrogolglycerides.

In another preferred embodiment, the permeation enhancer component further comprises 0.1 to 15%, and preferably 1-10% or 2-5% of a medium chain fatty acid. As used herein, the term medium chain fatty acid includes salts and derivatives thereof, such as sodium decanoate (also known as sodium caprate), sodium laurate, sodium caprylate, sodium N-[8-(2-hydroxybenzoyl)amino]caprylate (see e.g. Hess et al., Eur J Pharm Sci. (2005) 25:307-12), etc. Sodium decanoate and sodium caprylate are particularly preferred.

In another embodiment the permeation enhancer component further comprises 0.1 to 10%, preferably 0.2 to 5%, and more preferably 0.5 to 2.0% of a bile salt. Examples of suitable bile salts include sodium glycocholate, sodium deoxycholate, sodium taurocholate, sodium fusidate, sodium glycodeoxycholate, and sodium taurodihydrofusidate.

Certain polyoxyethylene sorbitan fatty acid esters, such as polysorbate 80, have been reported to inhibit P-glycoprotein-mediate efflux of drugs (see e.g. Cornaire et al. Arzneim—Forsch Drug Res (2000) 50:576-9). In a preferred embodiment, the composition further comprises an additional P-glycoprotein inhibitor such as, for example, d-alpha-tocopheryl polyethylene glycol 1000 succinate (TPGS), quinidine, and verapamil. In one embodiment, the P-glycoprotein inhibitor is TPGS in amounts of 1 to 20%, and preferably 2-10%. In another embodiment, the P-glycoprotein inhibitor is quinidine in amounts of 0.1 to 10%, and preferably 0.5 to 5%. The additional P-glycoprotein inhibitor may be incorporated into the permeation enhancer component and adsorbed onto the particulate carrier. Alternatively, it may be formulated such that its absorption through the intestine initiates prior to the vancomycin component of the composition, as described further below.

The permeation enhancer component of the composition is adsorbed onto a pharmaceutically-acceptable particulate carrier. Typically the particulate carrier constitutes 5 to 40 w/w % of the composition. Suitable carriers include starch, magnesium carbonate, kaolin, colloidal silica, silicon-dioxide, crosslinked polyvinylpyrrolidone, and calcium carbonate. Suitable methods for adsorbing liquids onto particulate carriers with the purpose of obtaining a solid dose formulation have been previously described (see e.g. Friedrich et al., Eur J Pharm Biopharm. (2006) 62:171-7, and references cited therein). In one exemplary method, a solution comprising the polyoxyethylene sorbitan fatty acid esters and any other ingredients of the permeation enhancer component is adsorbed onto the carrier by slow, drop-wise addition with blending and kneading. The permeation enhancer/carrier mixture is dried in an oven and pulverized into blendable powder. The enhancer-carrier powder is mixed with the vancomycin using a suitable blending procedure including additional processing additives (see Cote P et al., Pharm Dev Technol. (2006) 11:29-45) and compressed into tablets or filled into capsules, or other suitable solid oral dosage forms.

Targeted release technologies may be used to facilitate release of the vancomycin from the dosage form at the jejunum site of the small intestine, which we have found is its preferential site of absorption within the gastro-intestinal tract. In a preferred embodiment the dosage form has an external enteric coating that dissolves at approximately pH of 6.0 and above, to maximize drug release within the jejunum. A suitable enteric coating comprises anionic copolymers based on methacrylic acid and methyl methacrylate, such as EUDRAGIT® L 100 (available from Degussa Pharma Polymers, Germany).

In a further embodiment the dosage form comprises a P-glycoprotein inhibitor, which may also be present in the permeation enhancer component of the composition, that is immediately released into the intestine when the dosage form is dissolved. In one such embodiment, the vancomycin/permeation enhancer components of the composition form an inner core of the dosage form, and the P-glycoprotein inhibitor is present in an outer shell or coating surrounding the inner core. This allows a biphasic release, where the P-glycoprotein inhibitor is released first, followed by delayed release of the vancomycin. The outer shell may consist essentially of a P-glycoprotein inhibitor or may comprise a P-glycoprotein inhibitor mixed with a high-molecular weight polymer that controls the rate in which the outer shell dissolved by erosion or hydrolysis. Suitable high molecular weight polymers include gelatin, polylactic acid, polyglycolic acid, polycaprolactone and their combinations.

Another orally bioavailable vancomycin composition of the invention comprises at least 40%, 45%, 50%, 55%, 60%, 65%, 70%, or 75% vancomycin and 5-30%, or preferably 10-25% bile salts. Preferred bile salts are described above. The formulation may be administered orally as a liquid, or may be formulated into solid oral dosage forms. The formulation may further comprise a P-glycoprotein inhibitor in an amount effective to inhibit P-glycoprotein-mediated efflux of the vancomycin as described above.

The compositions of the invention, when orally administered to a mammal, yield a vancomycin bioavailability of at least 20%, and preferably at least 30%, 40%, or 50%. Oral bioavailability can be assessed using the Beagle dog, or equivalent model, wherein levels of plasma vancomycin from an orally administered composition are compared to levels obtained after i.v. administration using the calculation: BA=AUC_{PO}×Doseᵢᵥ/AUCᵢᵥ×Dose_{PO}; where BA=bioavailability and AUC=area under the plasma concentration-time curve.

The compositions are orally administered to a mammal that has a condition amenable to systemic treatment with vancomycin, such as for the treatment of serious or severe infections caused by susceptible strains of methicillin-resistant (beta-lactam resistant) staphylococci. In specific embodiments, the mammal is a human. In other embodiments, the mammal may be a livestock animal (horse, cow, pig, etc.) or a companion animal (e.g. dog, cat, etc.). For adult humans, the daily dose of absorbed vancomycin is approximately 2 grams. For pediatric administration, the daily dose is approximately 10 mg/kg.

In certain embodiments, a separately formulated P-glycoprotein inhibitor is administered prior to or together with the vancomycin composition. The composition may be provided in a kit with instructions on proper dosing. For example, the composition may be provided in a blister-pack kit, where one or more unit dosage forms are contained in a blister. The blister packaging may contain writing adjacent a blister or a row or column of blisters to indicate the proper timing of dosing. The kit may additionally contain a separately formulated P-glycoprotein inhibitor.

### EXAMPLE 1

### Drug Transport Studies

We investigated regional variation in permeation of vancomycin (VCM) across various segments of rat intestine and colon in vitro by mounting isolated segments of rat intestine and colon between horizontal modified Ussing chambers. The effects of drug concentration and of addition of a p-glycoprotein inhibitor or permeation enhancer(s) as formulation additives on VCM permeation were also studied in vitro using segments of rat jejunum.

Materials: Vancomycin hydrochloride (Spectrum Chemical, CA); 4-(2-hydroxyethyl)-1-piperazineethanesulfonic acid (HEPES) buffer (VWR, Brisbane, Calif.); bile salt, quinidine, and Sodium decanoate (all from Sigma, St. Louis, Mo.); Polysorbate-80 (Fluka Sigma-Aldrich, St. Louis, Mo.); Labrasol® (Caprylocaproyl macrogolglycerides) and Gelucire® 44/14 (Lauroyl macrogolglycerides) from Gattefosse, Paramus, N.J.; Vitamin E TPGS (d-α-tocopheryl polyethylene glycol 1000 succinate) from Eastman, N.J.

Methods: A modified Ussing system (Easy mount Ussing system, Physiological Instruments Inc., CA, Item # EM-CSYS-8) with eight sets of chambers was used for all in vitro studies; each set consisted of two parallel diffusion chambers, a heating block for temperature control, needle valves for gas flow adjustment and gas mixing, and Ag/AgCl voltage and current electrodes for measuring transepithelial voltage and for passing current.

Harvesting of required small intestinal or colon segments of a male rat was performed using experimental procedures described in the literature (Gotoh et al., J Biomol Screen (2005) 10:517-23). The harvested segments of small intestine or colon were mounted on sliders placed between the two horizontal chambers of a modified Ussing system.

VCM was dissolved in HEPES buffer pH 7.4 (Table 1) previously saturated with 100% O₂ or HEPES buffer containing additives selected from permeation enhancer(s) and/or an efflux transport inhibitor (Table 2) to obtain predetermined concentrations of drug and additive(s).

TABLE 1
Composition of HEPES Buffer (pH 7.4)

| Ingredient | Concentration in g/L |
|---|---|
| Sodium chloride | 135.0 |
| Potassium chloride | 5.4 |
| Calcium chloride | 1.2 |
| Magnesium chloride | 1.2 |
| Sodium hydroxide | 5.0 |
| HEPES* | 10.0 |
| Dextrose | 10.0 |

*HEPES = 4-(2-Hydroxyethyl)-1-piperazineethanesulfonic acid.

Drug transport across epithelial membranes of harvested rat small intestine and colon segments, in mucosal-to-serosal (M-to-S) direction, was studied using modified Ussing Chambers. Aliquots of HEPES buffer (5 ml) were initially added to both the mucosal and serosal chambers and allowed to equilibrate for 20 minutes. The HEPES buffer in the mucosal chamber was replaced by a study sample containing VCM alone or VCM and selected additive(s) in HEPES buffer, e.g., 5 mg/mL of VCM dissolved in HEPES buffer containing 10% w/w Labrasol®. Aliquots of 0.5 mL buffer solutions were removed periodically from serosal chambers, and replaced with equal volumes of fresh warm (37±2° C.) HEPES buffer previously saturated with 100% O₂. To study the effect of additives in the serosal chamber (i.e., quinidine and sodium decanoate), HEPES buffer containing the selected additive was placed in the serosal chamber and replaced with the same buffer composition during sampling.

Changes in transepithelial short-circuit current (in micro-Amps) and membrane resistance (in Ohms) as a function of time were monitored continuously during in vitro studies to serve as indicators of tissue viability and drug permeability, respectively. The buffer samples from the receptor chambers were analyzed for VCM content on a high pressure liquid chromatograph (HPLC; Hewlett Packard Model 1100 series).

The analysis of VCM samples was performed using a reverse phase gradient HPLC method, with a Hypersil BDS C18 column, 5 μm, 150×4.6 mm., mobile phase consisting of 5 mM potassium phosphate monobasic in water (pH 3) as solvent A and acetonitrile as solvent B at a flow rate of 1 ml/min (Farin et al., J Pharm Biomed Anal (1998) 18:367-72). The eluant was monitored by a UV/diode array detector at 280 nm.

To compare data obtained from different in vitro experiments, the apparent permeability coefficients were calculated using the equation:

$${P}_{app} = \frac{\frac{d Q}{d t}}{{C}_{0} \times A}$$

Where dQ/dt is the linear appearance rate of mass in receiver compartment, C₀ is the initial solute concentration in donor compartment, and A is the surface area (Luo et al., Drug Metab Dispos (2002) 30:763-70).

Results. Regional differences in permeation of VCM were observed across various segments of the gastrointestinal tract (g.i.t.). Based on calculated Pₐₚₚ values using in vitro data, the rate of M-to-S transport of VCM was found to be highest in jejunum, followed by colon and ileum, and lowest in duodenum. Cumulative mean values of VCM transported into the receptor compartment as a function of time are given in Table 3, and respective Pₐₚₚ values are given in Table 2.

TABLE 2
Apparent permeability coefficient (Pₐₚₚ) values for in vitro
transport studies on 5 mg/ml Vancomycin hydrochloride in the
presence and absence of enhancers across different
segments of rat intestine and colon, and across jejunum
in presence of various additives. Values
are an average of four replicates at each data point.

| | Pₐₚₚ Values for |
|---|---|
| | Mucosal-to-Serosal |
| | Transport |
| Vancomycin hydrochloride in HEPES buffer | (×10⁻⁶ m/sec) ± |
| with/without enhancers | Stdev (10⁻⁶) |
| HEPES buffer alone (Duodenum) | 0.20 ± 0.05 |
| HEPES buffer alone (Jejunum) | 0.62 ± 0.07 |
| HEPES buffer alone (Ileum) | 0.38 ± 0.05 |
| HEPES buffer alone (Colon) | 0.41 ± 0.07 |
| HEPES buffer alone (10 mg/mL) | 0.57 ± 0.13 |
| HEPES buffer alone (25 mg/mL) | 0.54 ± 0.69 |
| HEPES buffer alone (50 mg/mL) | 0.39 ± 0.47 |

HEPES buffer containing:

| | |
|---|---|
| 5% w/w Polysorbate-80 | 0.88 ± 0.09 |
| 5% w/w Gelucire ® 44/14 | 0.78 ± 0.03 |
| 10% w/w Labrasol ® | 0.36 ± 0.06 |
| 5% w/w Gelucire ® 44/14 + 5% w/w Labrasol ® | 0.71 ± 0.03 |
| 5% w/w Vitamin E TPGS + 5% w/w Labrasol ® | 0.56 ± 0.06 |
| 5% w/w Polysorbate-80 + 5% w/w Labrasol ® | 0.53 ± 0.06 |
| 0.15 mM Quinidine in mucosal chamber | 0.79 ± 0.08 |
| 0.15 mM Quinidine in serosal chamber | 2.06 ± 0.16 |
| 20% w/w Bile salts | 3.67 ± 0.58 |
| 1.0 mM sodium decanoate in mucosal chamber | 1.74 ± 0.10 |
| 1.0 mM sodium decanoate in serosal chamber | 0.60 ± 0.02 |

TABLE 3
Vancomycin transport in mucosal-to-serosal direction, across different
segments of rat intestine and colon (5 mg/ml vancomycin in HEPES buffer; total 25 mg)

| | Ave. Cumulative Amt VCM Released (μg) | | | | |
|---|---|---|---|---|---|
| SEGMENT | 0.5 hr | 1.0 hr | 1.5 hr | 2.0 hr | 2.5 hr |
| Duodenum | 0.00 +/− 0.00 | 0.00 +/− 0.00 | 0.40 +/− 0.80 | 2.08 +/− 0.87 | 3.51 +/− 1.34 |
| Jejunum | 0.00 +/− 0.00 | 1.06 +/− 0.95 | 3.73 +/− 0.61 | 6.94 +/− 1.07 | 11.08 +/− 1.08 |
| Ileum | 0.00 +/− 0.00 | 0.00 +/− 0.00 | 2.55 +/− 0.67 | 4.31 +/− 1.17 | 6.33 +/− 1.66 |
| Colon | 0.00 +/− 0.00 | 0.00 +/− 0.00 | 1.93 +/− 0.38 | 4.20 +/− 0.99 | 7.15 +/− 1.82 |

The results are in agreement with observations from in situ studies on VCM, in which lower segments of rat small intestine (jejunum, ileum) and colon were reported to be favorable sites for absorption (geary et al., supra; Yugi, J Pharm Sci Technol, Jpn, (1999) 59:103-12). Changes in transepithelial short-circuit current (micro-Amps) as a function of time were in the acceptable range for a viable tissues, while changes in transepithelial membrane resistance (Ohms) or potential differences across the two chambers were correlated with the rate of drug transport; these findings were consistent among the replicates.

Increasing the concentration of drug in the mucosal (donor) chamber increased M-to-S transport of VCM in a dose-dependent manner, as shown in Tables 2 and 4.

TABLE 4
Effect of drug concentration on vancomycin transport, in mucosal-to-serosal
direction, across rat jejunum (in HEPES buffer without enhancer)

| concentration | Ave. Cumulative Amt VCM Released (μg) | | | | |
|---|---|---|---|---|---|
| mg/ml | 0.5 hr | 1.0 hr | 1.5 hr | 2.0 hr | 2.5 hr |
| 5 | 0.00 +/− 0.0 | 1.06 +/− 0.95 | 3.73 +/− 0.61 | 6.94 +/− 1.07 | 11.08 +/− 1.08 |
| 10 | 0.00 +/− 0.0 | 2.76 +/− 0.69 | 6.88 +/− 0.98 | 12.86 +/− 1.23 | 20.77 +/− 1.90 |
| 25 | 0.00 +/− 0.0 | 2.90 +/− 1.82 | 7.99 +/− 5.71 | 21.56 +/− 3.84 | 50.91 +/− 6.58 |
| 50 | 0.97 +/− 1.37 | 7.94 +/− 5.19 | 22.26 +/− 4.61 | 46.13 +/− 1.25 | 69.54 +/− 4.20 |

Increased M-to-S transport of VCM was observed in vitro, when Quinidine was added to mucosal or serosal chambers. Addition of 0.15 mM Quinidine, a known P-glycoprotein inhibitor, to the serosal chamber caused a dramatic (3-fold) increase in M-to-S transport of VCM. Addition of 0.15 mM Quinidine to the mucosal chamber also increased M-to-S transport of VCM, but to a lesser extent. The data are represented in Table 5, and respective Pₐₚₚ values are given in Table 2. These observations are also in conformance with observations reported from in situ studies, in which M-to-S transport of VCM was increased in the presence of Verapamil and Cyclosporine-A, while Tetraethyl ammonium and Guanidine had no effect (Yugi et al., supra).

TABLE 5
Effect of 0.15 mM Quinidine in mucosal chamber (MC) or serosal chamber (SC)
on vancomycin transport, in mucosal-to-serosal direction, across rat jejunum (5 mg/ml
vancomycin in HEPES buffer; tissue slider area = 0.5 cm²)

| | Ave. Cumulative Amt VCM Released (μg) | | | | |
|---|---|---|---|---|---|
| Condition | 0.5 hr | 1.0 hr | 1.5 hr | 2.0 hr | 2.5 hr |
| No Q | 0.00 +/− 0.00 | 1.66 +/− 0.43 | 4.34 +/− 1.57 | 8.73 +/− 2.92 | 14.24 +/− 4.56 |
| Q in MC | 0.00 +/− 0.00 | 1.72 +/− 2.44 | 4.91 +/− 3.98 | 9.00 +/− 5.72 | 14.14 +/− 6.95 |
| Q in SC | 1.87 +/− 0.57 | 8.27 +/− 3.06 | 16.77 +/− 7.45 | 26.75 +/− 11.57 | 38.96 +/− 12.27 |

The formulation additives used in the present study include Labrasol® (Caprylocaproyl macrogol-8-glycerides), a novel emulsifier; Vitamin E TPGS® (α-tocopheryl polyethylene glycol 1000 succinate) and Gelucire® 44/14 (mixture of glycerol and PEG1500 esters of long fatty acids), both lipid-based amphiphilic carriers; and Polysorbate 80, a pharmaceutical emulsifier and solubilizer. Cumulative mean values of VCM transported to the receptor compartment with VCM alone and with VCM in the presence of the enhancers are shown in Table 6. The Pₐₚₚ values are given in Table 2.

TABLE 6
Effect of various permeation enhancers on vancomycin transport, in mucosal-to-
serosal direction, across rat jejunum

| | Ave. Cumulative Amt VCM Released (μg) | | | | |
|---|---|---|---|---|---|
| ENHANCER (w/w) | 0.5 hr | 1.0 hr | 1.5 hr | 2.0 hr | 2.5 hr |
| none (HEPES only) | 0.00 +/− 0.00 | 2.16 +/− 1.47 | 4.13 +/− 0.74 | 8.28 +/− 1.80 | 14.45 +/− 3.07 |
| 10% Labrasol ® | 3.65 +/− 1.22 | 3.72 +/− 2.58 | 5.52 +/− 1.36 | 7.48 +/− 1.11 | 9.93 +/− 1.23 |
| 5% Polysorbate-80 + 5% w/w | 0.00 +/− 0.00 | 2.36 +/− 0.16 | 3.85 +/− 0.39 | 6.05 +/− 0.80 | 10.12 +/− 1.39 |
| Labrasol ® | | | | | |
| 5% TPGS ® + 5% Labrasol ® | 0.00 +/− 0.00 | 1.26 +/− 1.78 | 3.48 +/− 1.13 | 6.40 +/− 2.08 | 10.03 +/− 3.20 |
| 5% Gelucire ® 44/14 | 0.40 +/− 0.79 | 3.72 +/− 0.40 | 6.81 +/− 1.07 | 10.87 +/− 1.53 | 14.29 +/− 2.36 |
| 5% Gelucire ® 44/14 + 5% | 0.81 +/− 1.40 | 3.56 +/− 0.25 | 6.63 +/− 1.67 | 9.58 +/− 1.81 | 13.78 +/− 5.10 |
| Labrasol ® | | | | | |
| 5% Polysorbate-80 | 0.00 +/− 0.00 | 2.33 +/− 0.24 | 5.69 +/− 1.64 | 10.15 +/− 2.82 | 15.99 +/− 3.24 |

Based on calculated Pₐₚₚ values of VCM, Polysorbate 80 and Gelucire® 44/14 at 5% w/w individually enhanced M-to-S transport of VCM across rat jejunum. For 5% w/w Labrasol® in combination with 5% w/w Gelucire® 44/14, the Pₐₚₚ value is slightly higher than that of VCM alone. For 5% w/w Labrasol® in combination with 5% w/w Vitamin E TPGS® or Polysorbate 80 slightly reduced VCM transport in the M-to-S direction was observed. VCM transport was lowest, when compared to VCM alone or in presence of other pharmaceutical excipients used in the study, in the presence of 10% w/w Labrasol®, with a Pₐₚₚ value of 0.36×10⁻⁶ cm/sec. In addition to promoting solubilization of poorly soluble drugs, Vitamin E TPGS®, Gelucire® 44/14, and Polysorbate 80 were also reported to modulate P-glycoprotein mediated efflux transport, and thus to increase the bioavailability of P-glycoprotein substrates such as paclitaxel, vinblastine, rhodamine 123, and digoxin (Dintaman et al., Pharm Res (1999) 16:1550-6; Comaire et al., supra; Sachs-Barrable et al., J Pharm Pharmaceut Sci, (2007) 10:319-331).

Addition of a high concentration of bile salts at 20% w/w to the mucosal chamber greatly enhanced M-to-S transport of VCM in vitro. The mechanisms by which bile salts influence transfer of solutes across the gastrointestinal epithelial membranes appear to be complex and have been the subject of many previous studies. Depending on the physiochemical properties of the drug under investigation and the interaction of the bile salts with the drug and epithelial cell membranes in the physiological environment; the mechanisms may involve micellar formation and/or alteration of the barrier function of the cell membrane, the mucus layer, or the tight junctions (Aungst (1996) supra; Kakemi et al., Chem Pharm Bull (1970) 18:275-80; Amidon et al., J Pharm Sci 1982; 71(1):77-84; Muranishi, Pharm Res (1985) 3:108-18; O'Reilly et al., Int J Pharm (1994) 109:147-54; Yamashita et al., J Pharm Sci (1990) 79:579-83; Werner et al., Pharm Res (1996) 13:1219-27).

TABLE 7
Effect of 20% bile salts on vancomycin transport, in mucosal-to-serosal
direction, across rat jejunum (5 mg/ml Vancomycin in HEPES buffer; tissue slider area = 0.5 cm²)

| | Ave. Cumulative Amt VCM Released (μg) | | | | |
|---|---|---|---|---|---|
| | 0.5 hr | 1.0 hr | 1.5 hr | 2.0 hr | 2.5 hr |
| HEPES only | 0.00 +/− 0.00 | 2.16 +/− 1.47 | 4.13 +/− 0.74 | 8.28 +/− 1.80 | 14.45 +/− 3.07 |
| 20% w/w bile salt | 0.00 +/− 0.00 | 5.40 +/− 2.26 | 18.19 +/− 7.43 | 37.58 +/− 9.54 | 66.39 +/− 18.91 |

Addition of sodium decanoate, 1.0 mM, to VCM in HEPES buffer in either mucosal chamber increases permeability to VCM. Addition of sodium decanoate to the serosal chamber had minimal effect on in vitro permeation of VCM, but addition to the mucosal chamber had a much greater effect.

TABLE 8
Effect of 1.0 mM sodium decanoate (SD) placed in mucosal chamber (MC) or
serosal chamber (SC) on vancomycin transport, in mucosal-to-serosal direction, across rat
jejunum

| | Ave. Cumulative Amt VCM Released (μg) | | | | |
|---|---|---|---|---|---|
| | 0.5 hr | 1.0 hr | 1.5 hr | 2.0 hr | 2.5 hr |
| No SD | 0.00 +/− 0.00 | 0.00 +/− 0.00 | 0.00 +/− 0.00 | 1.43 +/− 0.10 | 2.15 +/− 0.19 |
| SD in MC | 2.24 +/− 3.17 | 4.52 +/− 6.39 | 11.11 +/− 10.44 | 19.25 +/− 14.42 | 34.10 +/− 8.84 |
| SD in SC | 0.00 +/− 0.00 | 0.91 +/− 1.28 | 3.91 +/− 0.70 | 6.91 +/− 0.41 | 10.60 +/− 0.28 |

What is claimed is:

1. An orally bioavailable pharmaceutical composition comprising:
a) at least 40% (w/w) vancomycin;
b) less than 50% (w/w) of a permeation enhancer component comprising 0.1 to 10.0% (w/w) of a polyoxyethylene sorbitan fatty acid ester; and
c) a particulate carrier onto which the permeation enhancer component is adsorbed, wherein the permeation enhancer component increases the vancomycin apparent permeability coefficient across rat jejunal tissue in mucosal-to-serosal direction as measured in an in vitro Ussing system by at least 25%.

2. The composition of claim 1 wherein the carrier is selected from the group consisting of starch, magnesium carbonate, kaolin, colloidal silica, silicon-dioxide, crosslinked polyvinylpyrrolidone and calcium carbonate.

3. The composition of claim 1 wherein the permeation enhancer component further comprises a P-glycoprotein inhibitor selected from 1 to 20% (w/w) d-alpha-tocopheryl polyethylene glycol 1000 succinate (TPGS) and 0.1 to 10% (w/w) quinidine.

4. The composition of claim 1 wherein the permeation enhancer component further comprises 1-20% (w/w) macrogolglycerides.

5. The composition of claim 1 wherein the permeation enhancer component further comprises 1-20% (w/w) macrogolglycerides selected from the group consisting of lauroyl macrogolglycerides, caprylocaproyl macrogolglycerides, and stearoyl macrogolglycerides.

6. The composition of claim 1 wherein the permeation enhancer component further comprises 0.1 to 15% (w/w) of a medium chain fatty acid selected from the group consisting of sodium decanoate, sodium laurate, sodium caprylate.

7. The composition of claim 1 wherein the permeation enhancer component further comprises 0.5 to 2.0% (w/w) of a bile salt selected from the group consisting of sodium glycocholate, sodium deoxycholate, sodium taurocholate, sodium fusidate, sodium glycodeoxycholate, and sodium taurodihydrofusidate.

8. The composition of claim 1 in an enteric-coated unit dosage form.

9. The composition of claim 1 in an enteric-coated unit dosage form, wherein the enteric coating dissolves at approximately pH of 6.0 and above.

10. The composition of claim 1 in a capsule or tablet.

11. A method for treating a pathologic microbial infection in a mammal, the method comprising:
orally administering to the mammal an effective amount of the composition of claim 1.

12. The method of claim 11 wherein prior to the administering step, the mammal is administered a P-glycoprotein inhibitor in an amount effective to inhibit P-glycoprotein-mediated efflux of the vancomycin

13. A kit comprising the composition of claim 1.

14. The kit of claim 19 further comprising a P-glycoprotein inhibitor in a dosage form separate from the composition.

15. A pharmaceutical composition in an oral dosage form, said composition comprising at least 40% (w/w) vancomycin; and 5-30% (w/w) bile salts, wherein the composition has a vancomycin bioavailability of at least 40% when orally administered to a mammal.

16. The composition of claim 15 further comprising a particulate carrier onto which the bile salts are adsorbed.

17. The composition of claim 15 further comprising a P-glycoprotein inhibitor in an amount effective to inhibit P-glycoprotein-mediated efflux of the vancomycin.
